(19) United States
(12) Patent Application Publication
(10) Pub. No.: US 2020/0268271 A1
(43) Pub. Date: Aug. 27, 2020

(54) RR DAY DISPLAY FOR CARDIAC RHYTHMS

(71) Applicant: Cardiac Insight Inc., Kirkland, WA (US)

(72) Inventor: David Hadley, Woodinville, WA (US)

(21) Appl. No.: 16/285,995

(22) Filed: Feb. 26, 2019

(51) Int. Cl. A61B 5/0456; A61B 5/0402; A61B 5/0245; A61B 5/00; A61B 5/044

(52) U.S. Cl. CPC A61B 5/0456; A61B 5/0402; A61B 5/044; A61B 5/7425; A61B 5/0245

(57) ABSTRACT

Methods of processing electrocardiogram (ECG) data, and related systems, generate a beat-to-beat HR scatter plot/display for evaluation by a medical professional for diagnosis of cardiac irregularities. A method of processing ECG data includes processing ECG data recorded for a patient over a monitored time period to identify beat-to-beat intervals. Heart rates are calculated for the beat-to-beat intervals. The calculated heart rates include a respective heart rate for each of the beat-to-beat intervals. The calculated heart rates are displayed and/or plotted for an output period of time.

### BACKGROUND

Electrocardiography (ECG or EKG) is often used to assess the electrical and muscular functions of the heart using electrodes placed on the skin. The electrodes detect electrical variations that arise from heart muscle depolarization and repolarization during each heartbeat. EGG is a commonly employed method to assess the health of a person's heart.

In many instances, ECG is performed in a clinical setting, such as in a hospital or in a doctor's office. Performing ECG in a clinical setting, however, tends to discourage widespread application of ECG as a precautionary method for the detection of latent, undetected heart conditions, such as congenital heart defects. Moreover, time constraints in the clinical setting tend to limit the timespan over which the ECG is performed, which may inhibit detection of intermittently observable heart conditions.

Ambulatory physiological data monitors can be used to gather physiological data outside the clinical setting, thereby potentially enabling wider application of patient diagnoses than offered in the clinical setting. Ambulatory physiological data monitors also offer the potential of gathering physiological data over extended timespans that may not be practical in clinical settings.

The evaluation of ECG data typically requires time and effort on the part of a physician qualified to evaluate ECG data. Such a physician's time, however, is valuable and often in short supply due to competing demands on the physician's time. Accordingly, improvements in physician efficiency and productively with respect to evaluation of ECG data are of interest.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Methods of processing electrocardiography (ECG) data calculate and plot and/or display heart rates for beat-to-beat intervals. Each of the heart rates is calculated for a respective beat-to-beat interval (e.g., RR interval—where R is a point corresponding to the peak of the QRS complex of the ECG wave; and the RR interval is the time between successive Rs). In many embodiments, the calculated heart rates are displayed/plotted in a scatter-plot format. Advantageously, the resulting display and/or plot is indicative of whether the patient corresponding to the ECG data is suffering from a cardiac irregularity, such as, high idiopathic premature ventricular contractions (PVC) burden, paroxysmal atrial fibrillation (PAF), heart flutter, atrial bigeminy, and electrical conduction block. For many types of cardiac irregularities, the resulting display and/or plot exhibits a distinctive pattern of calculated beat-to-beat heart rates, such as a distinctive stratified pattern of beat-to-beat heart rates, which may be readily recognized by a medical professional with experience and/or training regarding the distinctive patterns and their associated cardiac irregularity. As a result, diagnostic efficiency, productivity, and/or accuracy may be improved.

Thus, in one aspect, a method of processing electrocardiogram (ECG) data is provided. The method includes: (a) processing ECG data recorded for a patient over a monitored time period to identify beat-to-beat intervals; (b) calculating heart rates for the beat-to-beat intervals, the heart rates comprising a respective heart rate for each of the beat-to-beat intervals; and (c) displaying and/or plotting the heart rates for an output time period.

In many embodiments of the method, the display and/or plot of the heart rates employs a time axis scale selected so that the heart rates for the output period of time are simultaneously displayed. The beat-to-beat heart rates can be simultaneously displayed for any suitable time period (e.g., at least 5 minutes, at least 30 minutes, at least 2 hours, at least 24 hours).

In many embodiments of the method, the identified beat-to-beat intervals include substantially all of the beat-to-beat intervals of the ECG data for the output time period. For example, in many embodiments, the identified beat-to-beat intervals include at least 90 percent of all beat-to-beat intervals of the ECG data for the output time period. In some embodiments, the identified beat-to-beat intervals include at least 99 percent of all beat-to-beat intervals of the ECG data for the output time period.

In many embodiments of the method, the heart rates are displayed and/or plotted as individual data points. For example, in many embodiments, the display and/or plot of the heart rates has a scatter plot format.

In many embodiments of the method, the ECG data is pre-recorded. For example, many embodiments of the method includes receiving the ECG data from an ambulatory monitor that recorded the ECG data.

In another aspect, a system for processing electrocardiogram (ECG) data includes at least one processor and a tangible memory device. The tangible memory device stores non-transient instructions executable by the at least one processor to cause the at least one processor to: (a) process ECG data recorded for a patient over a monitored time period to identify beat-to-beat intervals, (b) calculate heart rates for the beat-to-beat intervals, the heart rates comprising a respective heart rate for each of the beat-to-beat intervals, and (c) generate a display and/or plot of the heart rates for an output time period.

In many embodiments of the system, the display and/or plot of the heart rates employs a time axis scale selected so that the heart rates for the output period of time are simultaneously displayed. The beat-to-beat heart rates can be simultaneously displayed for any suitable time period (e.g., at least 5 minutes, at least 30 minutes, at least 2 hours, at least 24 hours).

In many embodiments of the system, the identified beat-to-beat intervals include substantially all of the beat-to-beat intervals of the ECG data for the output time period. For example, in many embodiments, the identified beat-to-beat intervals include at least 90 percent of all beat-to-beat intervals of the ECG data for the output time period. In some embodiments, the identified beat-to-beat intervals include at least 99 percent of all beat-to-beat intervals of the ECG data for the output time period.

In many embodiments of the system, the heart rates are displayed and/or plotted as isolated data points. For example, in many embodiments, the display and/or plot of the heart rates has a scatter plot format.

In many embodiments of the system, the ECG data can be pre-recorded. For example, many embodiments, the system is configured to receive the ECG data from an ambulatory monitor that recorded the ECG data.

In many embodiments, the system includes a suitable output device. For example, the system can include an output device via which the display and/or plot of the heart rates is generated.

For a fuller understanding of the nature and advantages of the present invention, reference should be made to the ensuing detailed description and accompanying drawings.

### BRIEF DESCRIPTION OF THE DRAWINGS

FIG. 1 shows a representative trace of ECG data.

FIG. 2 shows an example ten-minute interval summary plot of ECG data.

FIG. 3 shows a simplified schematic diagram of a method of processing ECG data and displaying and/or plotting beat-to-beat heart rates, in accordance with many embodiments.

FIG. 4 shows a representative RR interval for ECG data.

FIG. 5 illustrates processing ECG data and displaying and/or plotting beat-to-beat heart rates using the method of FIG. 3.

FIG. 6 shows a ten-minute interval summary plot of ECG data for a patient with high idiopathic premature ventricular contractions (PVC) burden.

FIG. 7 shows a beat-to-beat heart rate scatter plot for a patient with high idiopathic premature ventricular contractions (PVC) burden.

FIG. 8 shows a representative ten second trace of ECG data for a patient with paroxysmal atrial fibrillation (PAF).

FIG. 9 shows a ten-minute interval summary plot of ECG data for a patient with paroxysmal atrial fibrillation (PAF).

FIG. 10 shows a beat-to-beat heart rate scatter plot for a patient with paroxysmal atrial fibrillation (PAF).

FIG. 11 shows a representative ten second trace of ECG data for a patient with heart flutter.

FIG. 12 shows a ten-minute interval summary plot of ECG data for a patient with heart flutter.

FIG. 13 shows a beat-to-beat heart rate scatter plot for a patient with heart flutter.

FIG. 14 shows a representative ten second trace of ECG data for a patient with atrial bigeminy.

FIG. 15 shows a ten-minute interval summary plot of ECG data for a patient with atrial bigeminy.

FIG. 16 shows a beat-to-beat heart rate scatter plot for a patient with atrial bigeminy.

FIG. 17 shows a representative ten second trace of ECG data for a patient with ventricular bigeminy.

FIG. 18 shows a ten-minute interval summary plot of ECG data for a patient with ventricular bigeminy.

FIG. 19 shows a beat-to-beat heart rate scatter plot of ECG data for a patient with ventricular bigeminy.

FIG. 20 shows a system for processing ECG data and displaying and/or plotting beat-to-beat heart rates, in accordance with many embodiments.

FIG. 21 shows an ambulatory monitor, in accordance with many embodiments, adhered to the chest of a patient.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

Methods of processing electrocardiography (ECG) data, and related systems, for use in assessing whether a patient has a cardiac irregularity are described herein. The described methods and systems improve diagnostic efficiency by providing a display and/or plot in which many cardiac irregularities are indicated via a corresponding distinctive data pattern, which can be recognized by a medical professional with experience and/or training regarding the distinctive patterns and their associated cardiac irregularity. The methods and systems described herein for processing ECG data may be especially beneficial when used in conjunction with ECG data measured via ambulatory ECG monitoring.

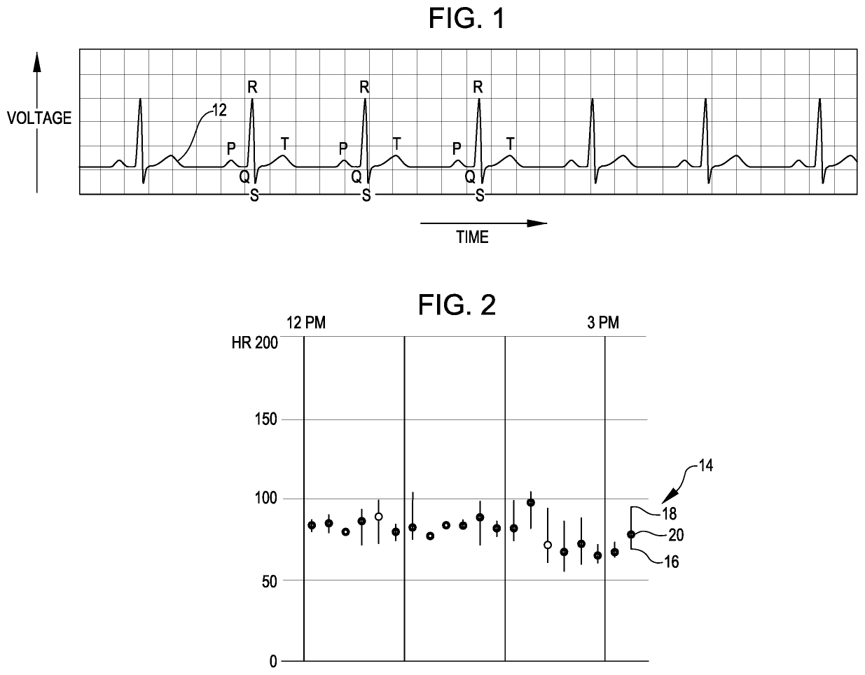

Referring now to the drawings, in which like reference numerals represent like parts throughout the several views, FIG. 1 shows a representative trace 12 of ECG data. As is known, an ECG measures the electrical activity of the heart. The trace 12 has a repeating pattern that starts with a P wave associated with right and left atrial depolarization, followed by a QRS complex associated with right and left ventricular depolarization, followed by an ST segment that reflects the current flow associated with phase 2 of ventricular repolarization, and then by a T wave that represents the current of rapid phase 3 ventricular repolarization. Deviation of an ECG trace from an ECG trace corresponding to a healthy heart can be indicative of one or more of many heart conditions.

Many heart conditions, however, are intermittent in nature. Accordingly, an ECG trace may only be intermittently indicative of a heart condition. ECG traces are also typically limited in the amount of time covered. For example, the ECG trace 12 of FIG. 1 only shows 7 heart beat cycles, which covers only a relatively small amount of time. Even at an assumed resting heart rate of 80 beats/minute, the ECG trace 12 only covers a little over 5 seconds of ECG data. For many intermittent heart conditions, it can be time consuming to diagnose such intermittent heart conditions via review of an ECG trace.

In an existing approach for diagnosing heart conditions via ECG data, each ten minute interval of the ECG data is sub-divided into twenty second segments and an average heart rate (HR) computed for each twenty second segment. The average HR of the twenty second segments, the highest average twenty second HR, and lowest average twenty second HR is then displayed and/or plotted for each ten minute interval of the ECG data. By consolidating each ten minute interval into a single display entity, the resulting display or plot can cover substantially more time than can be realistically covered by an ECG data trace. FIG. 2 shows an example ten-minute interval summary plot of ECG data covering about 3 hours. As shown, each display entity 14 includes a vertical line segment that extends upward from the lowest average twenty second HR 16 to the highest twenty second HR 18. Each display entity 14 also indicates the average HR 20 of the twenty second segments. A ten-minute interval summary plot is typically used to display ECG data in a form that allows simultaneous viewing of a substantially longer period of time as compared to an ECG trace. A ten-minute interval summary plot also serves to present the ECG data in a simplified form. By calculating an average HR for each twenty second interval and combining the average HR for each of the twenty second intervals into a ten minute display entity, the ten-minute interval summary plot effectively presents a summary of the ECG data for each ten-minute interval in a non-confusing, concise manner.

While the ten-minute interval summary plot of ECG data provides an effective concise summary of ECG data, the methods and systems described herein rely on a different approach for processing ECG data and presenting a graphical summary. The approach described herein presents a substantially greater amount of data that might otherwise serve to confound effective diagnosis of a cardiac condition but for the resulting display and/or plot exhibiting, for many cardiac irregularities, a corresponding distinctive pattern that can be readily recognized by a medical professional with experience and/or training regarding the distinctive patterns and their associated cardiac irregularity.

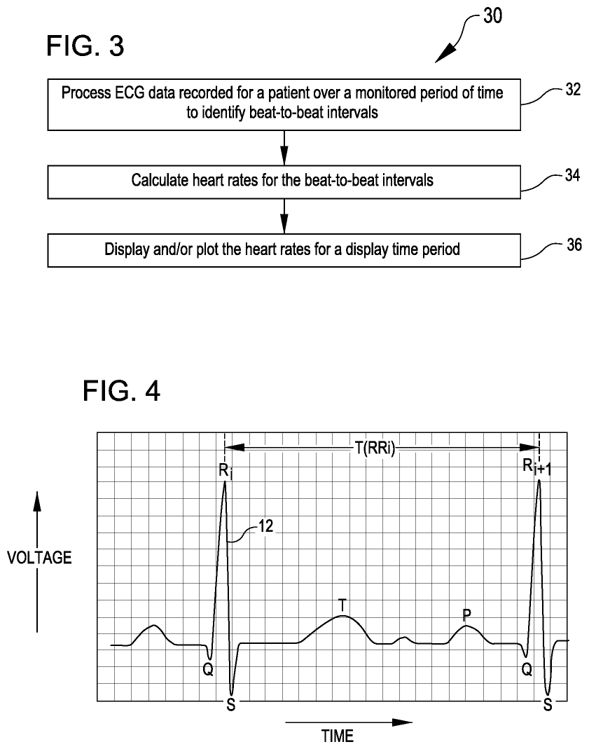

FIG. 3 shows a simplified schematic diagram of a method 30 of processing ECG data and presenting a resulting summary of the ECG data, in accordance with many embodiments. Any suitable device(s) and/or system(s), such as those described herein, can be used to practice the method 30. The method 30 includes processing ECG data recorded for a patient over a monitored period of time to identify beat-to-beat intervals (act 32). Any suitable reference point in an ECG trace can be used as starting point and a corresponding ending point for each beat-to-beat interval. For example, each beat-to-beat interval can be defined to correspond to an RR interval, a representative example of which is illustrated in FIG. 4 as t(RRᵢ). The ECG data can be processed to identify the instances in time at which each R point occurs using known approaches. A time span for each RR interval can then be calculated by subtracting the time at which the starting R point occurs from the time at which the ending R point occurs. For example, referring to FIG. 4, t(RRᵢ) can be calculated by subtracting the point in time at which Rᵢ occurs from the point in time at which Rᵢ₊₁ occurs.

In act 34, a heart rate (HR) is calculated for each beat-to-beat interval. For example, for HR expressed in terms of beats per minute and the units of the beat-to-beat interval being seconds, the HR for each interval can be calculated using equation 1.

HR(beat-to-beat interval)=60/(beat-to-beat interval) Equation 1:

In act 36, the calculated HRs are displayed (e.g., on a suitable display device) and/or plotted (e.g., as a paper hardcopy output) for a display time period. The calculated beat-to-beat heart rates can be simultaneously displayed for any suitable time period (e.g., at least 5 minutes, at least 30 minutes, at least 2 hours, at least 24 hours).

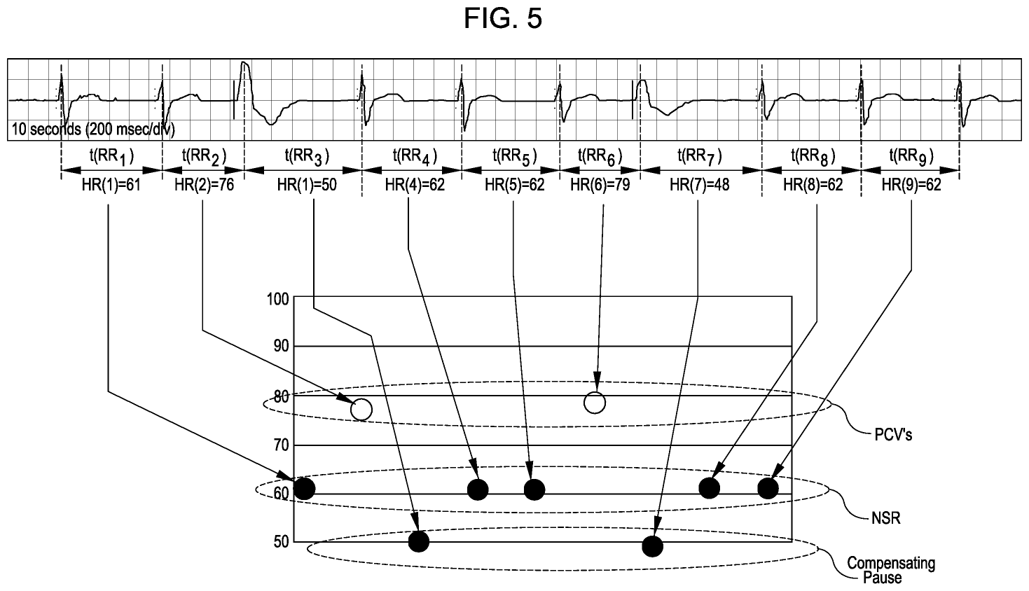

FIG. 5 illustrates processing ECG data and displaying and/or plotting beat-to-beat heart rates, from a patient with high idiopathic premature ventricular contractions (PVC) burden, using the method 30. For the ten second ECG data trace shown, the time span of each RR interval (e.g., t(RR₁) through t(RR₉)) is determined. A heart rate (HR) is then calculated for each RR interval. For example, t(RR₁) is equal to about 0.984 seconds, which corresponds to HR(1) being equal to about 61 beats/minute. The HRs (e.g., HR(1) through HR(9)) are then plotted in a scatter-plot format as illustrated in FIG. 5. The resulting plot highlights the occurrence of premature ventricular contractions in RR intervals two and six followed immediately by compensating pauses in RR intervals three and seven. The resulting stratification of the calculated HRs contributes to the generation of a recognizable pattern indicative of the patient having high idiopathic premature ventricular contractions (PVC) burden.

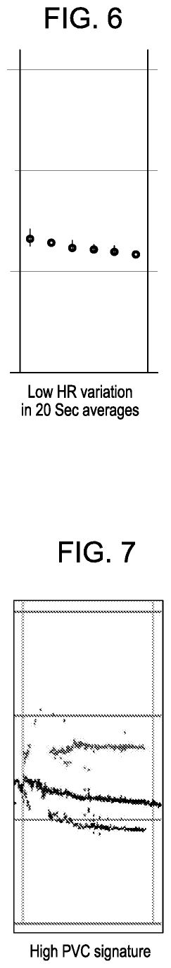

For many cardiac irregularities, the method 30 generates a resulting display and/or plot of beat-to-beat heart rates that can provide a more readily recognizable indication of the existence of the cardiac irregularity as compared to a ten-minute interval summary plot. For example, FIG. 6 shows a ten-minute interval summary plot of ECG data for a patient with high idiopathic premature ventricular contractions (PVC) burden. For comparison, FIG. 7 shows a beat-to-beat heart rate scatter plot for the same patient ECG. As can be seen, the resulting stratification of the scatter plotted RR-interval HRs readily show instances of premature ventricular contraction and compensating pauses that may be indicative of the patient having high idiopathic premature ventricular contractions (PVC) burden. In contrast, it may be less apparent looking at the ten-minute interval summary plot shown in FIG. 6 that the patient may have high idiopathic premature ventricular contractions (PVC) burden.

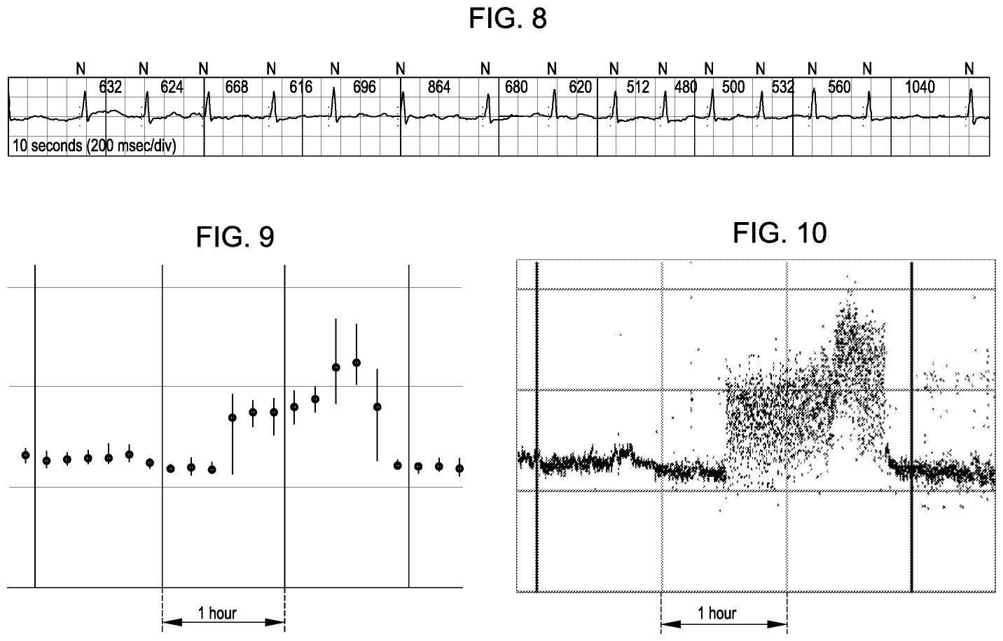

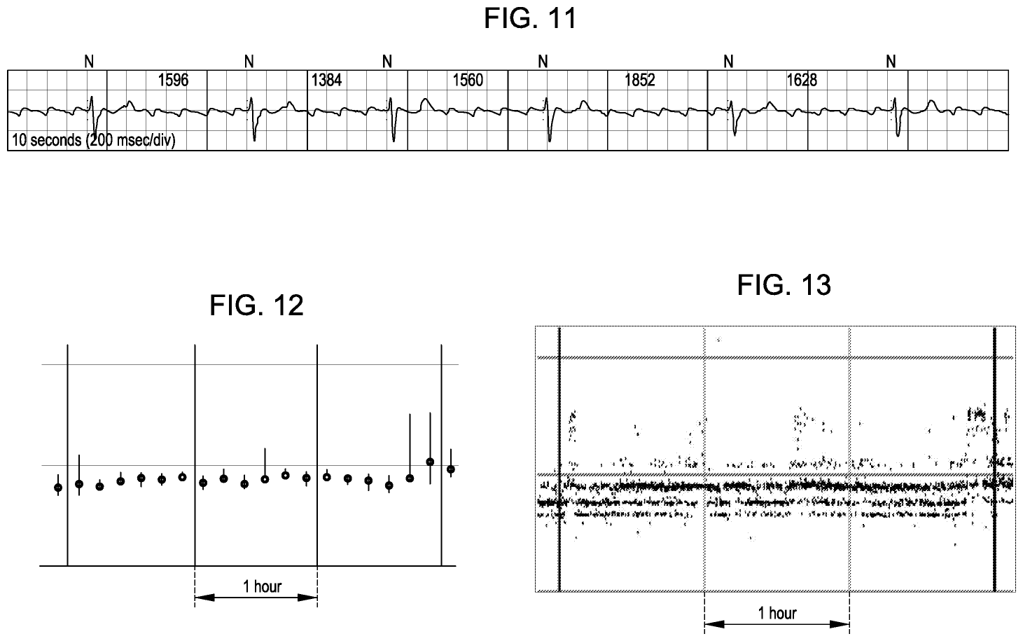

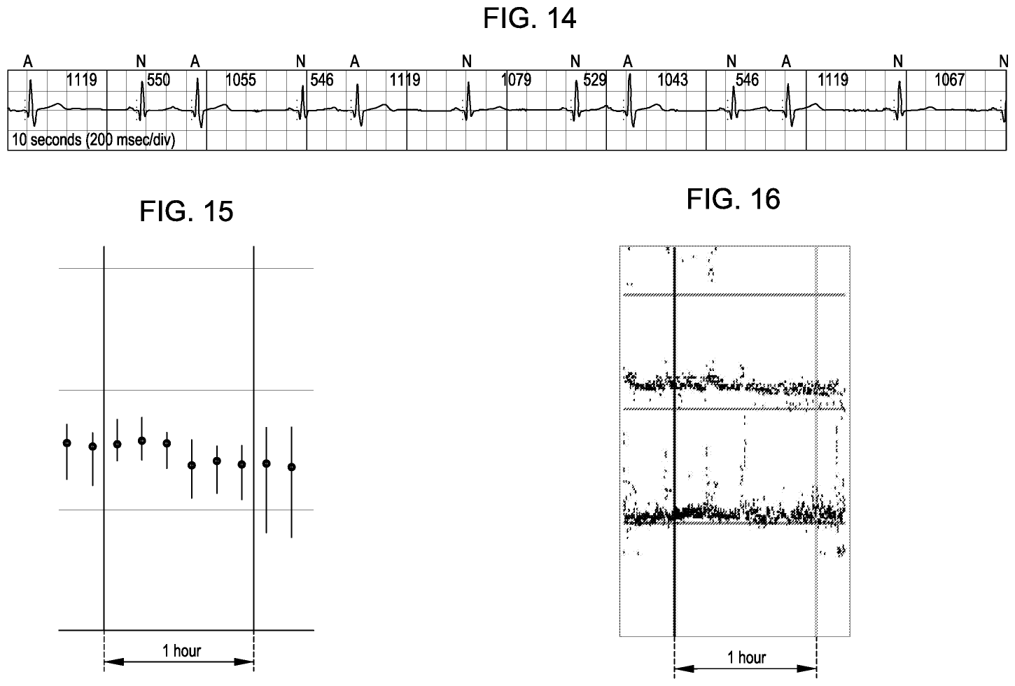

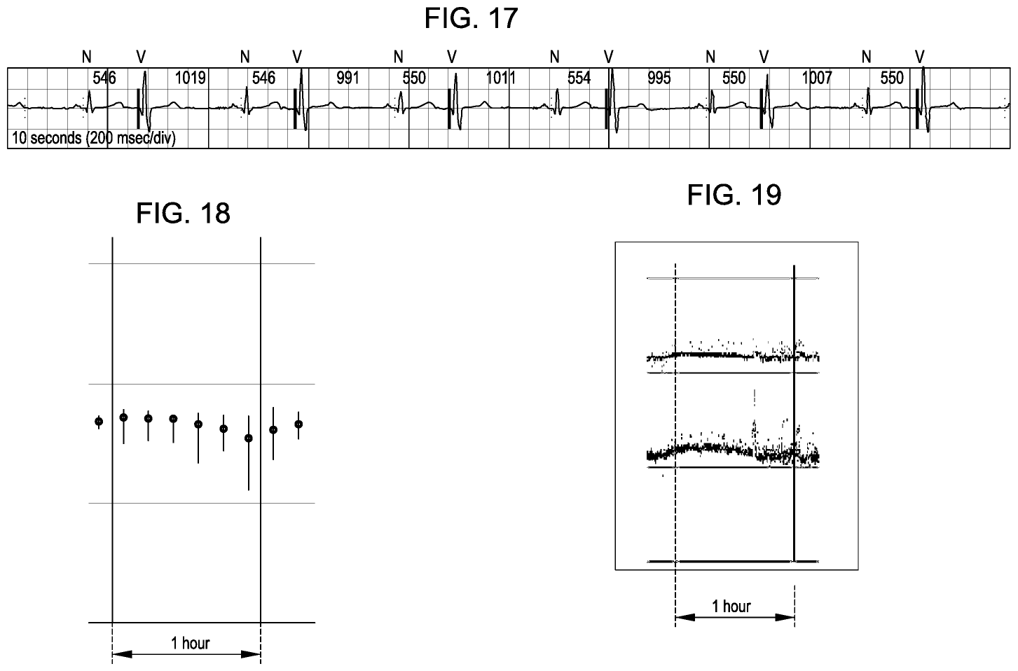

Additional cardiac irregularities for which the method 30 provides a resulting display and/or plot of beat-to-beat heart rates that exhibits a recognizable pattern include, but may not be limited to: paroxysmal atrial fibrillation (PAF), heart flutter, atrial bigeminy, and ventricular bigeminy. For example, FIG. 8 shows a representative ten second trace of ECG data for a patient with paroxysmal atrial fibrillation (PAF). FIG. 9 shows a ten-minute interval summary plot of ECG data for a patient with paroxysmal atrial fibrillation (PAF). FIG. 10 shows a beat-to-beat heart rate scatter plot for a patient with paroxysmal atrial fibrillation (PAF). FIG. 11 shows a representative ten second trace of ECG data for a patient with heart flutter. FIG. 12 shows a ten-minute interval summary plot of ECG data for a patient with heart flutter. FIG. 13 shows a beat-to-beat heart rate scatter plot for a patient with heart flutter. FIG. 14 shows a representative ten second trace of ECG data for a patient with atrial bigeminy. FIG. 15 shows a ten-minute interval summary plot of ECG data for a patient with atrial bigeminy. FIG. 16 shows a beat-to-beat heart rate scatter plot for a patient with atrial bigeminy. FIG. 17 shows a representative 10 second trace of ECG data for a patient with ventricular bigeminy. FIG. 18 shows a ten-minute interval summary plot of ECG data for a patient with ventricular bigeminy. FIG. 19 shows a beat-to-beat heart rate scatter plot for a patient with ventricular bigeminy.

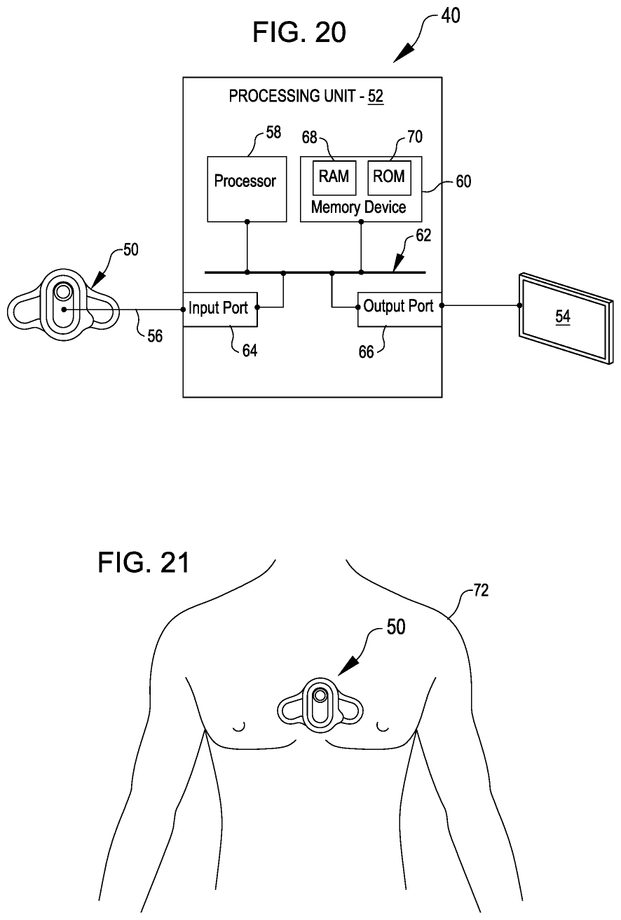

Any suitable system can be used to process ECG data to generate a beat-to-beat interval scatter plot using the method 30. For example, FIG. 20 shows a system 40 for processing ECG data and displaying and/or plotting beat-to-beat heart rates, in accordance with many embodiments. The system 40 includes an ambulatory monitor 50, a processing unit 52, and a output device 54. In many embodiments, the ambulatory monitor 50 is configured to be attached to a patient and record the ECG data for the patient over an extended period of time. The ECG data recorded by the monitor 50 can then be communicated to the processing unit 52 using a suitable approach (e.g., via direct suitable communication link, via suitable wireless transmission). In many embodiments, the monitor 50 can be detached from the patient and directly connected to the processing unit 52 via a download cable 56.

The processing unit 52 can have any suitable configuration. For example, in the illustrated embodiment, the processing unit 52 includes a processor 58, a memory unit 60, a communication bus 62, an input port 64, and an output port 66. The memory unit 60 includes a random access memory (RAM) device 68 and a read only memory (ROM) device 70. The ROM device 70 can store operating system instructions executable by the processor 58. The RAM device 68 can store program instructions executable by the processor 58 for accomplishing the acts of the method 30. In many embodiments, the RAM device 68 stores patient ECG data that was recorded by the monitor 50 and beat-to-beat interval HR data points calculated by the processor 58 for the patient ECG data. Each of the processor 58, the RAM device 68, the ROM device 70, the input port 64, and the output port 66 are communicatively connected via the communication bus 62. In many embodiments, following recordation of the patient ECG data, the monitor 50 is connected to the input port 64 via the download cable 56. The patient ECG data is then downloaded from the monitor 50 to the RAM device 68 via the communication bus 62. In some embodiments, the patient ECG data is downloaded from the monitor 50 to the processor 58, which then writes the patient ECG data to the RAM device 68. In many embodiments, the processor 58 executes program instructions stored in the memory device 60 that cause the processor 58 to process the patient ECG data to calculate the beat-to-beat HR data points in accordance with the method 30. In many embodiments, the processor 58 generates a suitable output that is communicated to the output device 54 and causes the output device 54 to display the beat-to-beat HR scatter plot and/or print the beat-to-beat HR scatter plot.

FIG. 21 shows the ambulatory monitor 50 adhered to the chest of a user 72. In embodiments described herein, the ambulatory monitory 50 is configured to record the patient ECG data for the patient 72 that is generated via two electrodes coupled with the user's skin. A person of skill will appreciate that the ambulatory monitor 50 can be configured with any suitable number of electrodes (e.g., two, three, four, five, six, or more electrodes) that are coupled with the user's skin to record the patient ECG data. The ambulatory monitor 50 can have any suitable configuration. For example, a suitable configuration for the ambulatory monitor is described in U.S. patent application Ser. No. 15/666,016, entitled Ambulatory Heart Monitor with Conductive Adhesive Connection to Electronics Module, the entire contents of which is hereby incorporated by reference.

The beat-to-beat HR scatter plots described herein provide numerous benefits. For example, the beat-to-beat HR scatter plots may be reviewed by a qualified medical professional to diagnose any of a number of different cardiac irregularities directly, reducing the need to review a more detailed report regarding the patient's ECG data and thereby improving efficiency and productivity. For example, atrial fibrillation may be confirmed via a quick viewing of the beat-to-beat HR scatter plot display/plot. PVC rhythms may be readily distinguishable from atrial rhythms via the beat-to-beat HR scatter plots described herein. Higher order rhythm complexities, with multiple ectopic foci, are manifest as multiple bands in the beat-to-beat HR scatter plot display/plot.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A method of processing electrocardiogram (ECG) data, the method comprising:
processing ECG data recorded for a patient over a monitored time period to identify beat-to-beat intervals;
calculating heart rates for the beat-to-beat intervals, the heart rates comprising a respective heart rate for each of the beat-to-beat intervals; and
displaying and/or plotting the heart rates for an output time period.

2. The method of claim 1, wherein the display and/or plot of the heart rates employs a time axis scale selected so that the heart rates for the output period of time are simultaneously displayed.

3. The method of claim 2, wherein the output time period is at least 5 minutes.

4. The method of claim 3, wherein the output time period is at least 30 minutes.

5. The method of claim 4, wherein the output time period is at least 2 hours.

6. The method of claim 5, wherein the output time period is at least 24 hours.

7. The method of claim 2, wherein the identified beat-to-beat intervals comprise at least 90 percent of all beat-to-beat intervals of the ECG data for the output time period.

8. The method of claim 7, wherein the identified beat-to-beat intervals comprise at least 99 percent of all beat-to-beat intervals of the ECG data for the output time period.

9. The method of claim 2, wherein the display and/or plot of the heart rates has a scatter plot format.

10. The method of claim 2, wherein the beat-to-beat intervals are RR intervals.

11. A system for processing electrocardiogram (ECG) data, the system comprising:
at least one processor; and
a tangible memory device storing non-transient instructions executable by the at least one processor to cause the at least one processor to:
process ECG data recorded for a patient over a monitored time period to identify beat-to-beat intervals;
calculate heart rates for the beat-to-beat intervals, the heart rates comprising a respective heart rate for each of the beat-to-beat intervals; and
generate a display and/or plot of the heart rates for an output time period.

12. The system of claim 11, wherein the display and/or plot of the heart rates employs a time axis scale selected so that the heart rates for the output time period are simultaneously displayed.

13. The system of claim 12, wherein the output time period is at least 5 minutes.

14. The system of claim 13, wherein the output time period is at least 2 hours.

15. The system of claim 14, wherein the output time period is at least 24 hours.

16. The system of claim 12, wherein the identified beat-to-beat intervals comprise at least 90 percent of all beat-to-beat intervals of the ECG data for the output time period.

17. The system of claim 16, wherein the identified beat-to-beat intervals comprise at least 99 percent of all beat-to-beat intervals of the ECG data for the output time period.

18. The system of claim 12, wherein the display and/or plot of the heart rates has a scatter plot format.

19. The system of claim 12, configured to receive the ECG data from an ambulatory monitor that recorded the ECG data.

20. The system of claim 12, wherein the beat-to-beat intervals are RR intervals.
